(19) United States
(12) Patent Application Publication
(10) Pub. No.: US 2023/0187078 A1
(43) Pub. Date: Jun. 15, 2023

(54) SCALABLE RISK PREDICTION USING PRESCRIPTION DATA

(71) Applicant: VANDERBILT UNIVERSITY, Nashville, TN (US)

(72) Inventors: Colin Walsh, Nashville, TN (US); Michael Ripperger, Nashville, TN (US); Qingxia Chen, Nashville, TN (US); Drew Wilimitis, Nashville, TN (US)

(21) Appl. No.: 18/066,821

(22) Filed: Dec. 15, 2022

Related U.S. Application Data
(60) Provisional application No. 63/289,757, filed on Dec. 15, 2021.

(51) Int. Cl. G16H 50/30; G16H 20/10

(52) U.S. Cl. CPC G16H 50/30; G16H 20/10

(57) ABSTRACT

The present disclosure presents systems and methods of predicting overdose risk using prescription data. One such method comprises analyzing, by a computing device, a plurality of document datasets, where the document datasets comprising prescription data for a controlled substance, hospital discharge data, socioeconomic data, and vital statistics data of a subject; predicting an overdose risk for the subject within a set period of time based on the plurality of document datasets; aggregating overdose risk predictions involving the controlled substance for a plurality of subjects within a geographic region; and/or calculating an overall overdose risk involving the controlled substance for the geographic region.

### CROSS-REFERENCE TO RELATED APPLICATION

This application claims priority to co-pending U.S. provisional application entitled, “Scalable Risk Prediction Using Statewide Prescription Data,” having Ser. No. 63/289,757, filed Dec. 15, 2021, which is entirely incorporated herein by reference.

### BACKGROUND

The link between the current opioid epidemic in the United States and the over-prescribing of opioid pain relievers (OPRs) has been well established. Over-prescribing and OPR-related harms were first observed in the 1990s and some states including Tennessee (TN) have experienced higher rates of prescribing and the subsequent harms. Near the opioid prescribing peak in 2010, TN providers wrote more OPR prescriptions than there were residents in the state. Between 2014 and 2018, OPR-related deaths rose 49% to an annual cost of 1307 lives. The United States meanwhile has seen a near-universal adoption of prescription drug monitoring programs (PDMPs) with intentions to combat the opioid epidemic by monitoring prescribing histories, informing providers, and identifying concerns with varying success. Although PDMPs have seldomly been used to predict imminent risk at the patient level, prevention at the practice, county, or regional levels might be possible if accurate algorithms are developed, validated, and implemented.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a block diagram illustrating an exemplary computing system or device that can be utilized for systems and methods of the present disclosure.

FIG. 2 is a flow chart illustrating an exemplary method of determining a probability of an overdose of a controlled substance in accordance with various embodiments of the present disclosure.

FIG. 3 shows a table of characteristics of weak learner models and ensemble models for fatal and nonfatal overdose predictions for an experimental study of an exemplary method in accordance with various embodiments of the present disclosure.

FIG. 4 shows a table depicting risk concentration of the ensem bled fatal and nonfatal prediction models for an experimental study of an exemplary method in accordance with various embodiments of the present disclosure.

FIG. 5 shows a table of performance metric values for an experimental study of an exemplary method in accordance with embodiments of the present disclosure.

FIG. 6 shows charts of performance metric values for fatal and non-fatal overdose predictions for varying number of partitions of training data.

FIGS. 7 and 8 show a chart of predictive features by mean rank of importance for fatal and nonfatal opioid overdose models respectively.

### DETAILED DESCRIPTION

Overdose prevention is currently directed after harm has already occurred—for example, basing “high impact area” designations on deaths that have already occurred, not those we seek to prevent. Accordingly, the present disclosure presents a method of predicting overdose risk using prescription data, hospital discharge data, and/or vital statistics data. An exemplary method and related systems utilizes prescription data and basic clinical data including patient demographics and clinical diagnoses. In various embodiments, weak learner based ensemble learning techniques can be performed. For example, extreme case imbalance can be handled through novel machine learning approaches in ensemble learning to generate multiple “weak learners” that are subsequent ensembles into a meta model that performs best at the prediction task at hand, such as overdose risk prediction for a controlled substance. In various embodiments, the disclosed analytics can be used as a service to guide overdose risk prevention at state- or federal-levels and/or in a risk prediction model software for use in pharmacy settings, among other possible uses.

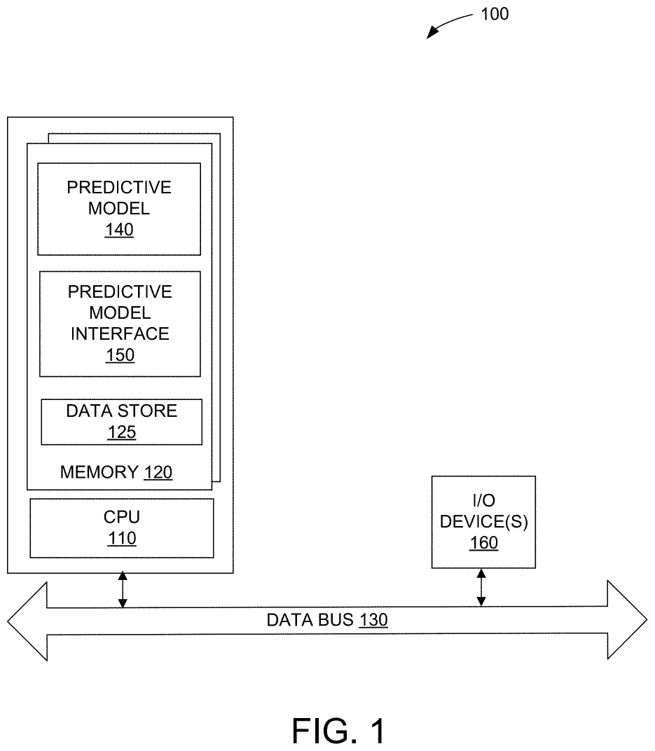

FIG. 1 is a block diagram illustrating an exemplary computing system or device 100 that can be utilized for systems and methods of the present disclosure. Computing system 100 includes at least one processor, e.g., a central processing unit (CPU), 110 coupled to memory elements 120 through a data bus 130 or other suitable circuitry. Computing system 100 stores program code within memory elements 120. Processor 110 executes the program code accessed from memory elements 120 via the data bus 130. In one aspect, computing system 100 may be implemented as a computer or other data processing system, including tablets, smartphones, or server computers that are accessed using browsers at client computers. It should be appreciated, however, that computing system 100 can be implemented in the form of any system including a processor and memory that is capable of performing the functions described within this disclosure.

Memory elements 120 include one or more physical memory devices such as, for example, a local memory and one or more storage devices. Local memory refers to random access memory (RAM) or other non-persistent memory device(s) generally used during actual execution of the program code. Storage device may be implemented as a hard disk drive (HDD), solid state drive (SSD), or other persistent data storage device. Computing system 100 may also include one or more cache memories (not shown) that provide temporary storage of at least some program code in order to reduce the number of times program code must be retrieved from storage device during execution.

Stored in the memory 120 are both data and several components that are executable by the processor 110. In particular, stored in the memory 120 and executable by the processor 110 are code for a predictive model of an overdose risk for a controlled substance (140) and code for interfacing with the predictive model and outputting a predictive outcome from the predictive model (150). Also stored in the memory 120 may be a data store 125 and other data. The data store 125 can include an electronic repository or database relevant to predictive model results. In addition, an operating system may be stored in the memory 120 and executable by the processor 110. In an embodiment, predictive model data are stored in the data store 125, such as model parameters.

For example, a predictive model may include a digitally constructed model of a probability of overdose for a controlled substance. In this context, the model refers to an electronic digitally stored set of executable instructions and data values, associated with one another, which are capable of receiving and responding to a programmatic or other digital call, invocation, or request for resolution based upon specified input values, to yield one or more stored output values that can serve as the basis of computer-implemented recommendations, output data displays, or machine control, among other things. Persons of skill in the field find it convenient to express models using mathematical equations, but that form of expression does not confine the models disclosed herein to abstract concepts; instead, each model herein has a practical application in a computer in the form of stored executable instructions and data that implement the model using the computer. The model may include a model of past events on the one or more fields, a model of the current status of the one or more fields, and/or a model of predicted events on the one or more fields. Model and field data may be stored in data structures in memory, rows in a database table, in flat files or spreadsheets, or other forms of stored digital data.

Input/output (I/O) devices 160 such as a keyboard, a display device, and a pointing device may optionally be coupled to computing system 100. The I/O devices may be coupled to computing system 100 either directly or through intervening I/O controllers. A network adapter may also be coupled to computing system to enable computing system to become coupled to other systems, computer systems, remote printers, and/or remote storage devices through intervening private or public networks. Modems, cable modems, Ethernet cards, and wireless transceivers are examples of different types of network adapter that may be used with computing system 100.

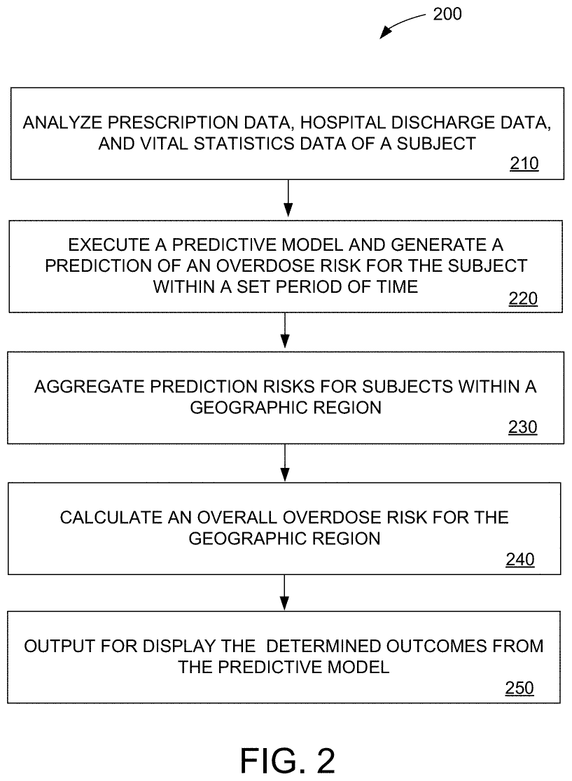

FIG. 2 is a flow chart illustrating an exemplary method 200 that may be implemented by computing system 100 described with reference to FIG. 1. Computing system 100 may execute, or include, an architecture as described generally with reference to FIG. 2. For example, the exemplary method includes modeling a probability of an overdose of a controlled substance. In block 210, the computing system may analyze, by a computing device or system 100, a plurality of document datasets comprising prescription data for the controlled substance, hospital discharge data, and vital statistics data of a subject. In block 220, the computing system 100 may execute a predictive model and generate a prediction of an overdose risk for the subject within a set period of time based on the plurality of document datasets. Accordingly, in block 230, overdose risk predictions involving the controlled substance are aggregated, by the computing system 100, for a plurality of subjects within a geographic region. In block 240, an overall overdose risk involving the controlled substance for the geographic region is calculated, by the computing system 100. In various embodiments, the overdose risk is related to the opioid crisis and may include a risk of a non-fatal overdose and a risk of a fatal overdose related to an opioid drug. Lastly, in block 250, the determined outcome(s) of the predictive model is output for display, such as the calculated overall overdose risk for the geographic region and/or the individual overdose risk for the subject.

Severely affected by the opioid crisis, Tennessee (TN) has already linked its controlled substance PDMP (II-V scheduled and gabapentin) to statewide mortality data and hospital discharge data. In one study, researchers at TDH and Vanderbilt University Medical Center (VUMC) partnered to develop and validate the first scalable predictive models from statewide datasets in TN for the related but disparate outcomes: (1) fatal and (2) nonfatal opioid overdose.

In 2019, a federal investigation led by the Department of Justice (DOJ) uncovered fraud and inappropriate opioid prescribing in TN and resulted in the arrests of multiple physicians, pharmacists, and other health professionals. Such measures relied upon descriptive analytics for harms that had already occurred years prior. While monitoring and descriptive analytics may provide a lens into the current state of the opioid epidemic, they cannot identify the next patient, practice, or community at risk. A goal of the present disclosure is to supplement these traditional epidemiological methods of identifying and characterizing risk with precise and automated predictive models.

For example, in a non-limiting embodiment, prescription data is ingested and preprocessed to calculate Morphine Milligram Equivalents by day and in total, presence or absence of overlapping benzodiazepine prescriptions, drug class categories using Anatomic Therapeutic Classification categories. Additionally, clinical data can be ingested and used to calculate the presence or absence of diagnostic classes, such as substance use disorders, mood disorders, heart failure diagnoses, etc., as per Clinical Classification Software categories. Further, publicly available data on syringe programs, buprenorphine providers, pain specialty clinics, etc. can be integrated to best predict risk of i) fatal and ii) non-fatal opioid-related overdose risk.

An exemplary method of the present disclosure can predict an overdose risk using data present at the moment a prescription is filled, and the method is scalable to other prediction targets and might include novel relevant data sources with model updating. In various embodiments, prescription data can be combined with clinical discharge and socioeconomic data to predict fatal and nonfatal opioid overdose within a period of time, such as 30 days, of a controlled substance prescription fill. Partitioning and ensembling the data allows the computer system 100 to use all study data despite computational limits. Accordingly, an overdose risk can be modeled at the prescription level, making these models applicable to any individual prescription with historical data. Aggregating these predictions enables risk to be calculated at varying levels of detail for better informed public health decision-making (e.g., at the local, county, and regional levels).

For testing and evaluation purposes, an experimental study was performed for an experimental method/system of the present disclosure. As data sources for supplying input data, a controlled substance monitoring database provided prescription data for subject(s); a hospital discharge data system (HDDS) data provided hospital discharge data for the subject(s), and death certificates provided vital statistics data of the subject(s) for a period spanning from the beginning of 2012 through the end of 2017. Publicly available socioeconomic indicators relating to health, healthcare utilization, and treatment access were compiled and mapped to either ZIP codes or counties.

The following were mapped to residential ZIP codes: Area Deprivation Index (ADI); statistics on employment from the U.S. Census Bureau; and Medication-Assisted Treatment (MAT) locations including buprenorphine providers, methadone clinics, and Opioid Treatment Programs (OTPs) from data aggregated by the Tennessee Department of Health (TDH). TN age-adjusted morbidity rates from TDH; the Tennessee Vulnerability Index (TVI) from TDH; statistics on income, poverty, college education, crowding, and private insurance from the American Community Survey (ACS); Rural-Urban Continuity Codes (RUCC) from the U.S. Department of Agriculture; the Social Vulnerability Index (SVI) from the Centers for Disease Control and Prevention (CDC); and Anti-Drug Abuse Coalition services from TDH were mapped to individual counties.

Outcomes of interest in the experimental study include fatal and nonfatal opioid-related overdose events that occurred within 30 days of a controlled substance prescription fill. The 30-day time window was chosen after plotting the accumulation of overdoses over time after a prescription fill. Fatal and nonfatal overdoses were identified consistent with methods used by TDH in their annual Prescription Drug Overdose Reports. Fatal overdoses were identified from TN death certificates using International Classification of Disease, revision 10 (ICD-10) codes. Nonfatal overdoses were identified in the HDDS with specified opioid-related diagnostic codes.

For the study, predictive modeling details were as follows: (1) a vector of socioeconomic indicators were established based on a patient's last reported location from the prescription data (i.e., provided via PDMP from the time of the previous prescription; (2) cumulative number of prior medications, diagnostic codes, and hospital visits (by type a patient has accumulated thus far) were counted; and (3) age, sex, and derived variables that represent a patient's prescription history for controlled substances were added.

Variables chosen included the sums of distinct practitioners, distinct pharmacies, distinct hospital identifiers, total prescriptions, total morphine milligram equivalents, short/long-acting OPR prescriptions, overlapping OPR and benzodiazepine prescriptions, prior medications for opioid use disorder, and opioid-naIve prescriptions as defined as not having an OPR prescription within the last 45 days. Race and ethnicity were not explicitly represented in the models used in the study. Modeling at the prescription level was done to create time-dependent and granular risk predictions which could then be aggregated to practice, pharmacy, local, county, and regional levels.

Patient linkage across the datasets relied on TDH-determined master patient indexing. Only records with valid person identifiers were retained, and records determined to be related to a nonhuman patient (e.g., veterinary prescription records) were removed. Hospital records from the HDDS were limited to verified inpatient encounters.

Precise area deprivation index (ADI) and Rural-Urban Continuity Codes (RUCC) features were developed from the minimum, maximum, and mean values of each ZIP code. Other ZIP code features were developed from county data using the TN county that contained the majority area of each ZIP code. OTP and methadone clinic availability were modeled using a 60-mile radius, representing a practical range for driving a normal distance in TN (90-120 minutes driving time).

To reduce the dimensionality of PDMP and HDDS features, prior medications and diagnoses were grouped to higher-order categories using the National Drug File-Reference Terminology (NDFFT), Pharmacologic Classes and Clinical Classification Software (CCS), Level 2 groupings from National Drug Codes (NDCs) and International Classification of Disease, revision 10, Clinical Modification (ICD-10-CM) codes. In total, 342 features were used for model training after this dimensionality reduction and only entries in patient records prior to prediction dates were used.

For predictive model training, the input data was separated into 75% training, 5% development, and 20% testing partitions to ultimately derive one model for fatal overdose and one model for nonfatal. All prescriptions in the data that were associated with an individual were added together to only form one set to prevent leak between training and testing within individuals. Models were trained in the training set and then calibrated, ensembled, and evaluated in the development set.

The training set was equally divided into 10 smaller training partitions or subsets due to computational limits. To help combat case imbalance, all cases and their associated records were added to each training set, but only 10% of all the controls from the entire training set were included in an individual training set (i.e., only one training set contained any one control). Ten random regression forest “weak learners” were then developed from the training subsets using the ranger R package (for random forest modeling) with an estimated response variance splitting criteria. To help limit memory consolidation, 200 trees were used for each random forest. In total, 20 random forests were developed from the 20 training subsets-10 for each of the 2 outcomes.

During training, each training subset itself was split into a 90% training set and a 10% testing set to allow predictions to be made for each case. Each case was placed in the testing set of each subset exactly one time which guaranteed all case data were used in training and at least one prediction for each associated record was generated. After the weak learners were ensembled and calibrated in the development set, the resulting ensembled models were validated in a final held-out testing set.

A development set consisting of 5% of the data was reserved to correct the miscalibrations from the under-sampled controls in the training subsets. 7 methods of ensembling and calibration were compared. Either the minimum, maximum, mean, or median prediction was taken from the 10 weak learner predictions and passed through logistic calibration, or the 10 weak learner predictions were used as inputs for ridge regression, random forest, or penalized regression (LASSO).

Logistic calibration, when applied, was defined by training a univariate logistic regression in the calibration set where the sole predictor was the aggregate in question (e.g., max) and the outcome was either fatal or nonfatal overdose. The resulting generalized linear models along with the aggregation methods were then considered as ensemblers. The more complex ensembling methods trained multivariate models using the 10 weak learners as predictors. Random forest was used for comparison for 2 types of penalized logistic regression: L1-regularized (LASSO) and L2-regularized (RIDGE) regression. All resulting models were expected to be calibrated as they were either trained on the calibration set or calibrated via logistic calibration.

Final ensembled and calibrated algorithms were then tested on the test set. Weak learners were tested on the calibration set. It is noted that no additional calibration was performed on the test set, making it a pure test of calibration as well as discrimination.

Performance metrics for evaluating the prediction models included area under the receiver operating curve (AUROC), area under the precision recall curve (AUPRC), and risk concentration. Risk concentration was performed by dividing the predictions from the test set into 10 quantiles and calculating the proportion of all the cases those quantiles held. Calibration was assessed using Spiegelhalter z-test. The ridge regression ensembles were further assessed for performance differences by subgroups consisting of race, ethnicity, and gender as determined by hospital records as well as age and RUCC codes from residential ZIP codes for urbanicity/rurality. To test how performance varied when the number of partitions in the training set was changed, additional models were trained using N=5 or N=15 and compared using AUPRC. For both fatal and nonfatal overdose, each feature was ranked by taking the mean of the important values from the 10 weak learners—determined by the variance of responses from each random forest.

For the experimental results, study data included 71,479,191 controlled substance prescriptions across 3,041,668 TN patients. As sourced from hospital records, when available: 1,409,556 (46.3%) patients were Female; 958,440 (31.5%) patients were Male; and 673,672 (22.1%) patients were Unknown. Patients by coded race showed 7,104 (0.23%) patients were Asian-American; 360,314 (11.8%) patients were Black; 704 (0.023%) patients were Native American; 20,147 (0.66%) patients were Other; 1,851,324 (61.0%) patients were White; and 802,075 (26.4%) patients were Unknown. Patients by coded ethnicity also showed 16,061 (0.53%) patients were Hispanic; 2,064,654 (67.8%) patients were non-Hispanic; and 960,953 (32.0%) patients were Unknown. Within 30 days, 2,574 fatal overdoses occurred after 4,912 (0.0069%) prescriptions and 8,455 nonfatal overdoses occurred after 19,460 (0.027%) prescriptions. Nearly 60% of all fatal and nonfatal overdoses in the data occurred within 30 days of a prescription.

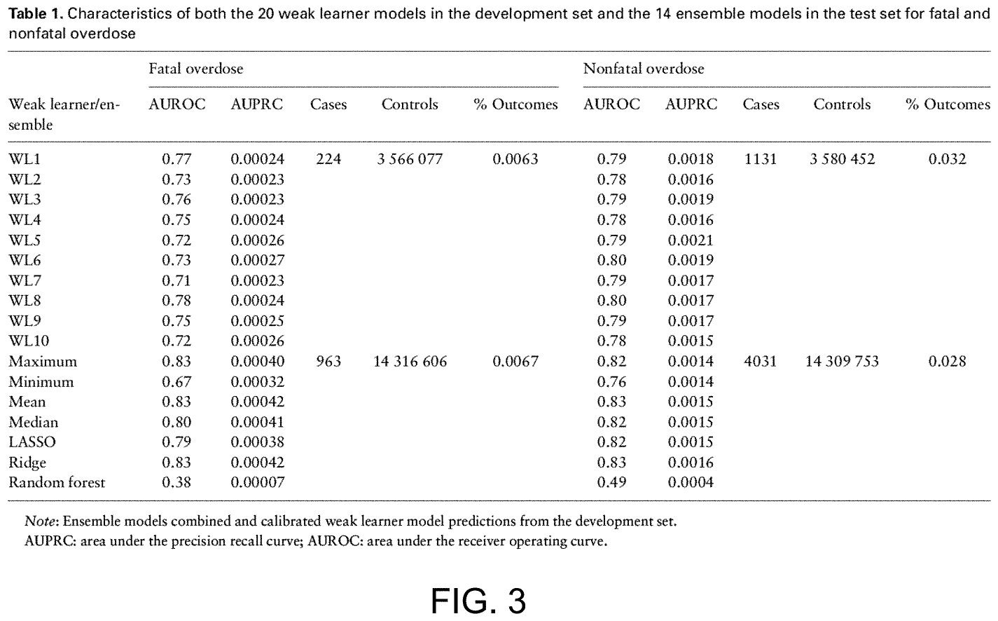

Both the fatal and nonfatal weak learner models had similar prevalence rates throughout the training set and showed consistent AUROC and AUPRC values when applied to the development set, as shown in FIG. 3 (Table 1). AUROC was useful to compare these models simply despite having known problems when assessing absolute performance with case imbalance. The total number of cases and controls in the training set were 3725 and 53 591 596 (0.0069%) for fatal and 14 695 and 53 580 626 (0.027%) for nonfatal overdose.

Discrimination varied by ensembling method when applied to the test set for both fatal and nonfatal overdose, illustrated in FIG. 3. Averaging or selecting the minimum or maximum predictions from the 10 weak learner models for both fatal and nonfatal produced similar results to using more complex methods of aggregation (e.g., ridge, LASSO). Random forest performed worse compared to other methods of aggregation. The top 2 performing ensembles, mean and ridge regression, were further evaluated in the risk concentration and calibration analyses.

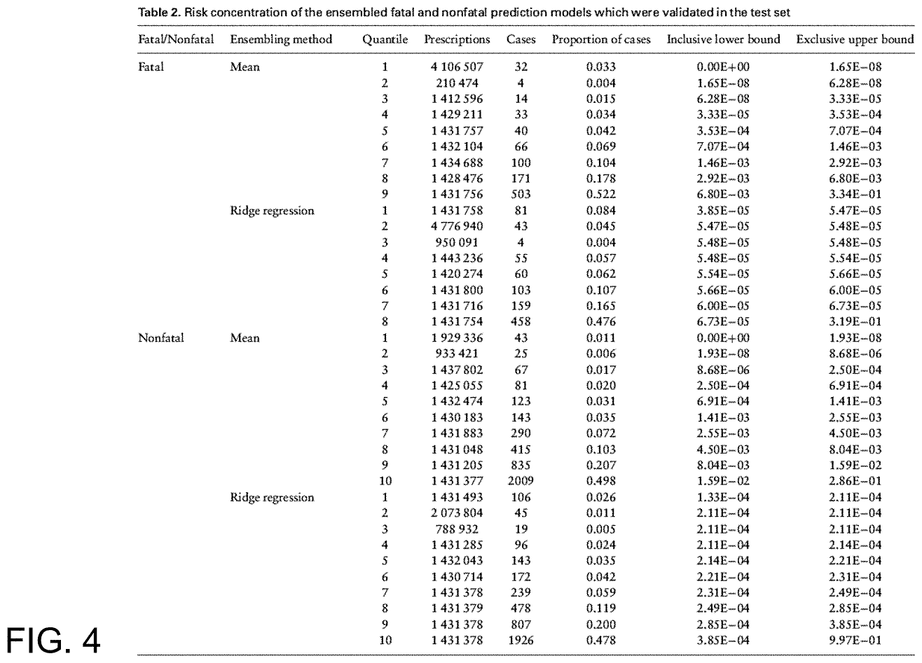

Risk concentration showed that, in the test set, the mean and ridge regression ensembling methods concentrated 47-52% of the overdose outcomes within the top quantiles of predicted probabilities, as shown in FIG. 4 (Table 2). Both top quantiles contained 10% of the test set predictions. Overlapping quantiles where the predictions had the same values were combined as seen by the number of prescriptions in the first quantile of the fatal mean ensembling method.

Calibration measured the degree to which the predictions reflected the true outcome prevalences. The ensembled models predicting fatal overdose showed nonsignificant calibration from mean ensembling and significant calibration from ridge regression as indicated by the nonsignificant Spiegelhalter z-test. The ensembled models for nonfatal overdose showed better calibration for ridge regression than for mean ensembling although both were nonsignificantly calibrated, as shown in Table I below. The ridge regression ensembling method was subsequently used to analyze performance variations by subgroups.

TABLE I

| Ensembled model | Brier score | Intercept | Slope | Sz | Sp |
|---|---|---|---|---|---|
| Fatal mean | 0.0001305 | −5.5329 | 0.6205 | −191.59 | 0.00 |
| Fatal ridge regression | 0.0000673 | −0.3313 | 0.9599 | 1.55 | 0.120 |
| Nonfatal mean | 0.0004239 | −4.0305 | 0.7625 | −272.14 | 0.00 |
| Nonfatal ridge regression | 0.0002923 | −1.7524 | 0.7942 | −9.34 | 0.00 |

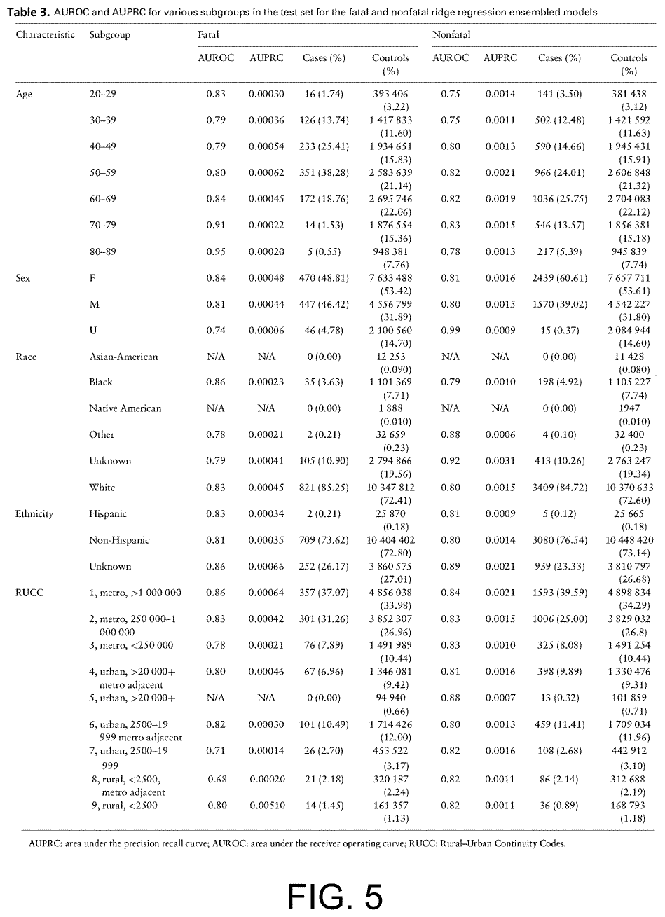

Subgroup performance differences and partition variation. Both the fatal and nonfatal ridge regression ensembles were tested on subgroups in the test set. AUROC and AUPRC values varied by subgroup in age, sex, race, ethnicity, and RUCC values of residential ZIP codes, as shown in FIG. 5 (Table 3). Case and control percentages among the subgroups also varied.

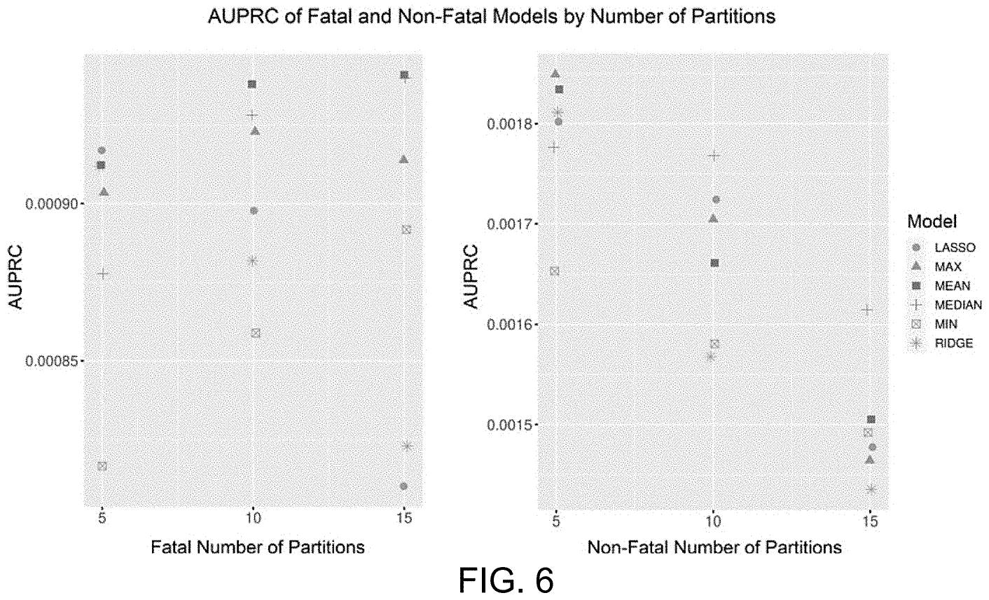

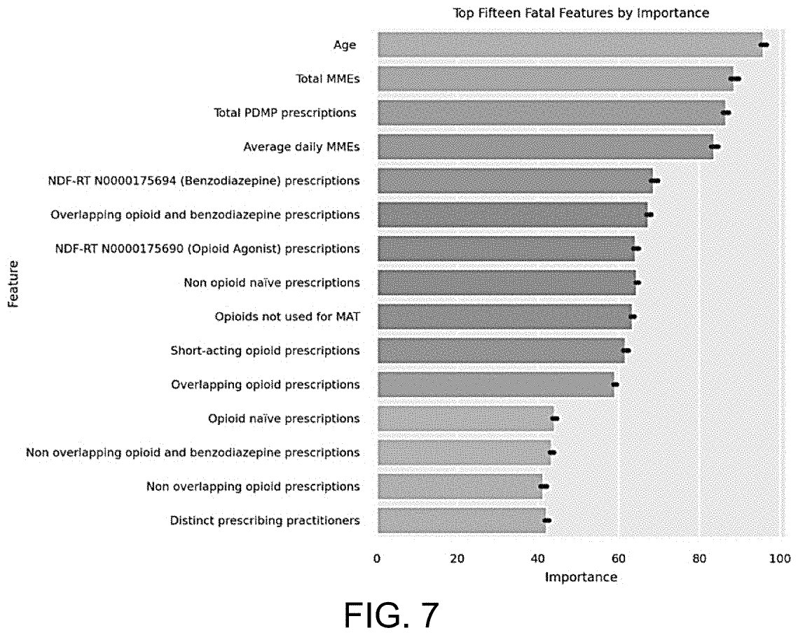

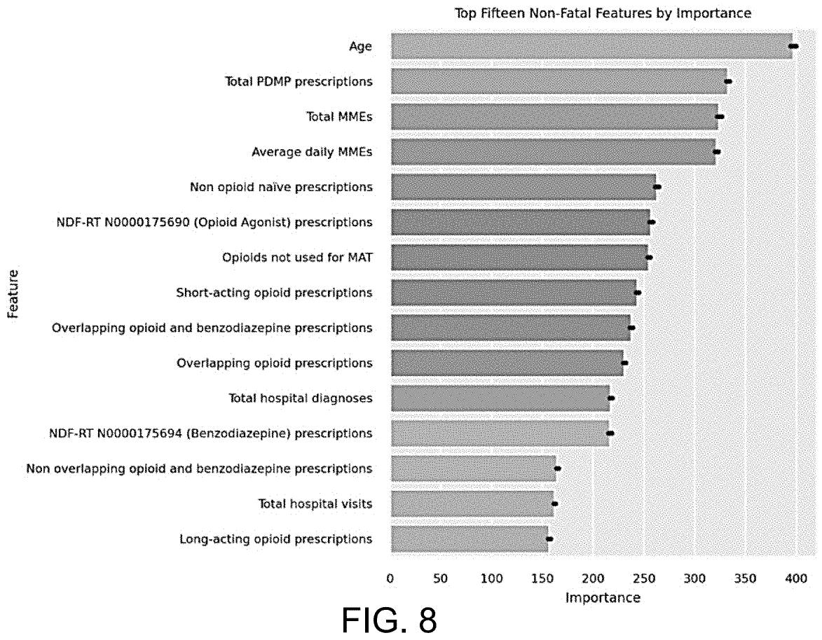

Repeating the modeling experiments for N=5 and N=15 showed no differences in AUPRC values when the number of partitions was changed, as shown in FIG. 6. Absolute change by partition choice was minimal as evidenced by the small absolute differences in y-axes shown (e.g., <0.0001 change in AUPRC by number of folds for the fatal model). The top 15 model features from the 10 weak learner models for fatal and nonfatal overdose were determined by ranking their mean response variances, as shown in FIGS. 7 and 8). Correspondingly, twelve features were within the top 15 of both the fatal and nonfatal overdose models.

The foregoing experimental study and its results support the validity of combining prediction data (e.g., via statewide PDMP data) with clinical discharge, vital statistics data, and/or socioeconomic data to predict fatal and nonfatal opioid overdose within 30 days of a controlled substance prescription fill. Partitioning and ensembling the data allowed us to use all study data despite computational limits. By modeling risk at the prescription level, making these models are applicable to any individual prescription with historical data. Aggregating these predictions enables risk to be calculated at varying levels of detail for better informed public health decision-making.

AUROCs and AUPRCs of the fatal and nonfatal models in the development set improved in the test set after ensembling, as indicated in FIG. 3. Risk concentration analyses consistently captured half the outcomes of interest in the top quantiles of risk, as indicated in FIG. 4. Given the presence of case imbalance, the highest risk quantiles may enable TN to focus prevention efforts more efficiently.

The subgroup performance analysis showed that the ridge regression models resulted in disparate performance in terms of AUPRC and AUROC for race and age despite small absolute AUPRC differences, as indicated in FIG. 5. Case imbalance may be driving these differences. Correcting performance differences is necessary for accurately assessing risk in the state. When the number of training partitions was varied, AUPRCs varied minimally if at all, as shown in FIG. 6.

In the fatal overdose model, the top predictors were face valid as known risk factors for opioid-related overdose, as shown in FIG. 7. The total quantity of controlled substances prescribed was close to the top of the list. Notably, overlapping benzodiazepine prescriptions were more important in the prediction of fatal opioid-related overdose than nonfatal. Multidrug combinations have been known to play a large role in the fatality potential of opioid-related overdoses and benzodiazepines have a synergistic respiratory depressant effect when taken with opioids.

Informatics implications of the experimental study include the importance of partitioning and sampling to lessen overfitting in settings with high stake, but rare (at state scale), outcomes. Efforts to predict risk at an actionable timepoint, for example, a prescription fill event, do not obviate aggregating risk analyses to levels relevant for public health intervention such as the community and regional levels. U.S. states have long implemented PDMPs, but most have not disseminated predictive modeling approaches at this scale. Characterizing OPR risk might inform better prevention, as the overdose crisis varies considerably across state lines.

Several attributes of this overdose modeling problem increased its complexity. First, extreme case imbalance resulted from the rarity of fatal and nonfatal overdoses at statewide scale—prevalence less than a fraction of 1%. Second, person disambiguation in data that were manually entered by pharmacists (into the Controlled Substance Monitoring Database (CSMD)) resulted in reliance on constructed, probabilistic patient mapping indices. Third, CSMD data contain human and nonhuman controlled substance prescription data. Removing those prescriptions known to be nonhuman was straightforward but ensuring nonhuman data are not miskeyed as human was not.

The training-development-test framework in the experimental study enriched case data in the presence of case imbalance without discarding valuable noncase comparator data. The weak learner approach overcame computational constraints which may apply to other groups attempting similarly scaled experiments. The study included the use of comprehensive real-world data derived from statewide operational datasets. Vital records, validated by medical examiners, and certified hospital discharge records were used to ensure modeling decisions reflected the implementation environment and were responsive to public health informatics requirements for overdose prevention. In addition, the outcome ascertainment strategy did not seek to determine if the patient's last prescription was the actual cause of the overdose outcome nor was it used in those risk calculations. Historical clinical and demographic information were also added to these models from batched HDDS data. Calculating risk in real-time remains challenging given the additional steps necessary to incorporate data entered close to the time of prediction.

In brief, exemplary methods and systems of the present disclosure ingested and analyzed prescription data, hospital discharge data, and death certificates from vital records, which were linked to socioeconomic indicators, to produce ensem bled overdose prediction models. As demonstrated by an experimental study, the predictive models were able to granularly predict fatal and nonfatal overdose risk within 30 days of receiving a controlled substance prescription. These predictions when aggregated may lead to more informed prevention efforts, including prevention efforts at the local, county, and regional levels.

Certain embodiments of the present disclosure can be implemented in hardware, software, firmware, or a combination thereof. If implemented in software or firmware, as one exemplary embodiment, the prediction risk models and related methods can be stored in a computer-readable medium (e.g., memory) that is executed by a suitable instruction execution system. If implemented in hardware, as in an alternative embodiment, the prediction risk models and related methods can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

In the context of this document, a “computer-readable medium” can be any means that can contain, store, communicate, or transport the program for use by or in connection with the instruction execution system, apparatus, or device. The computer readable medium can be, for example but not limited to, an electronic, magnetic, optical, electromagnetic, infrared, or semiconductor system, apparatus, or device. More specific examples (a nonexhaustive list) of the computer-readable medium would include the following: an electrical connection (electronic) having one or more wires, a portable computer diskette or drive (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical).

It should be emphasized that the above-described embodiments of the present disclosure are merely possible examples of implementations, merely set forth for a clear understanding of the principles of the disclosure. Many variations and modifications may be made to the above-described embodiment(s) without departing substantially from the principles of the present disclosure. All such modifications and variations are intended to be included herein within the scope of this disclosure.

1. A method comprising:
analyzing, by a computing device, a plurality of document datasets, the document datasets comprising prescription data for a controlled substance, hospital discharge data, socioeconomic data, and vital statistics data of a subject;
predicting, by the computing device, an overdose risk for the subject within a set period of time based on the plurality of document datasets;
aggregating, by the computing device, overdose risk predictions involving the controlled substance for a plurality of subjects within a geographic region; and
calculating, by the computing device, an overall overdose risk involving the controlled substance for the geographic region.

2. The method of claim 1, wherein the computing device implements a supervised learning method to perform the analyzing and predicting operations.

3. The method of claim 2, wherein the supervised learning method comprises a weak learner based ensemble learning method.

4. The method of claim 1, wherein the set period of time commences from filling of a prescription for the controlled substance.

5. The method of claim 4, wherein the set period of time is 30 days.

6. The method of claim 1, wherein the controlled substance is an opioid drug.

7. The method of claim 1, wherein the overdose risk prediction for the subject comprises a non-fatal overdose risk prediction.

8. The method of claim 1, wherein the overdose risk prediction for the subject comprises a fatal overdose risk prediction.

9. A system comprising:
at least one processor; and
memory configured to communicate with the at least one processor, wherein the memory stores instructions that, in response to execution by the at least one processor, cause the at least one processor to perform operations comprising:
analyzing a plurality of document datasets, the document datasets comprising prescription data for a controlled substance, hospital discharge data, socioeconomic data, and vital statistics data of a subject;
predicting an overdose risk for the subject within a set period of time based on the plurality of document datasets;
aggregating overdose risk predictions involving the controlled substance for a plurality of subjects within a geographic region; and
calculating an overall overdose risk involving the controlled substance for the geographic region.

10. The system of claim 9, wherein the analyzing and predicting operations are performed using a supervised learning method.

11. The system of claim 10, wherein the supervised learning method comprises a weak learner based ensemble learning method.

12. The system of claim 9, wherein the set period of time commences from filling of a prescription for the controlled substance.

13. The system of claim 12, wherein the set period of time is 30 days.

14. The system of claim 9, wherein the controlled substance is an opioid drug.

15. The system of claim 9, wherein the overdose risk prediction for the subject comprises a non-fatal overdose risk prediction.

16. The system of claim 9, wherein the overdose risk prediction for the subject comprises a fatal overdose risk prediction.

17. A non-transitory computer-readable medium having instructions stored therein, wherein the instructions, when executed by a processor, cause the processor to:
analyze a plurality of document datasets, the document datasets comprising prescription data for a controlled substance, hospital discharge data, socioeconomic data, and vital statistics data of a subject;
predict an overdose risk for the subject within a set period of time based on the plurality of document datasets;
aggregate overdose risk predictions involving the controlled substance for a plurality of subjects within a geographic region; and
calculate an overall overdose risk involving the controlled substance for the geographic region.

18. The non-transitory computer-readable medium of claim 17, wherein the analyzing and predicting operations are performed using a supervised learning method.

19. The non-transitory computer-readable medium of claim 18, wherein the supervised learning method comprises a weak learner based ensemble learning method.

20. The non-transitory computer-readable medium of claim 17, wherein the set period of time commences from filling of a prescription for the controlled substance.
